(19) United States
(12) Patent Application Publication
(10) Pub. No.: US 2008/0058670 A1
(43) Pub. Date: Mar. 6, 2008

(54) Animal Condition Monitor

(75) Inventor: Christopher E. Mainini, Knoxville, TN (US)

Correspondence Address: PITTS AND BRITTIAN P C, P O BOX 51295, KNOXVILLE, TN, 37950-1295, US

(73) Assignee: Radio Systems Corporation, Knoxville, TN (US)

(21) Appl. No.: 11/462,750

(22) Filed: Aug. 7, 2006

(51) Int. Cl. A61B 5/00

(52) U.S. Cl. 600/549

(57) ABSTRACT

An animal condition monitor. The animal condition monitor measures diagnostic parameters from the animal and determines whether the parameters indicate that the animal is at risk of a specified condition, such as heat stroke, heat exhaustion, hypothermia, or hyperthermia. The condition assessment is considered in light of a profile containing information about the age/health of the animal. When warranted, the animal condition monitor provides notification of the condition of interest.

### CROSS-REFERENCE TO RELATED APPLICATIONS

Not Applicable.

### STATEMENT REGARDING FEDERALLY SPONSORED RESEARCH OR DEVELOPMENT

Not Applicable.

### BACKGROUND OF THE INVENTION

1. Field of Invention

The invention relates to animal monitoring devices. More specifically, this invention relates to an electronic animal monitoring device for communicating information about conditions pertaining to an animal.

2. Description of the Related Art

Domesticated animals fill a wide variety of roles in the lives of people everywhere. Some animals are found in working or sporting positions while others enjoy positions as pets offering companionship. As an example, dogs are common pets and are also widely used as working dogs (guard dogs, drug dogs, seeing-eye dogs) and as sporting dogs (trackers and retrievers).

Technology currently exists to monitor environmental conditions and physiological conditions. Currently, there are no monitoring devices that provide an indication that an animal may be at risk of a foreseeable condition such as hypothermia, hyperthermia, heat exhaustion, or other related conditions. Animals lack the ability to communicate their condition so the ability to alert someone about the risk is an important feature of a condition monitoring device. While the condition of heat exhaustion is discussed as an exemplary condition, other conditions have similar serious consequences and are of equal concern.

By way of example, just as humans are subject to heat exhaustion, so are their animal companions. Every animal is at risk of injury or death resulting from heat exhaustion and heat stroke. Obviously, active animals have elevated body temperatures that are not quickly dissipated in warmer months and climates, but there are other contributing causes. For example, many of the working dogs and pets are transported in a vehicle with their owners and are sometimes subjected to elevated temperatures in the vehicles when left inside without air conditioning. There are a number of factors that increase the animal's susceptibility to heat exhaustion including the color and thickness of the coat, the physical condition, and the age of the animal.

While well-meaning, many owners lack knowledge about the symptoms associated with heat exhaustion and are unable to recognize the danger to the animal until too late. Observable symptoms include excessive panting, inner-ear flushing, weakness, staggering, and fainting. Even when symptoms are present, it is likely difficult for an animal owner to identify the cause just based on visual observation and the owner is unlikely to have a thermometer or other diagnostic equipment available. While recognizing the signs of heat exhaustion is important, it is generally accepted that prevention is the best medicine.

### BRIEF SUMMARY OF THE INVENTION

One embodiment of an animal condition monitor is shown and described. The animal condition monitor measures diagnostic parameters from the animal and the environment and determines whether the parameters indicate that the animal is experiencing a selected condition. The particular condition being monitored is based on the types of sensors provided in the animal condition monitor.

The animal condition monitor includes a controller that provides the processing and control to evaluate the input conditions and generates an indication in the event that the risk of a monitored condition occurring within the animal exceeds a selected level. One set of inputs to the controller is generated from one or more environmental condition sensors (e.g., temperature, humidity, barometric pressure, etc.). Another set of inputs to the controller comes from one or more physiological condition sensors (e.g., activity level, distance traveled, heart rate, respiration, blood pressure, body temperature, etc.). In the context of the present invention, the term physiological condition sensor refers to sensors that obtain information related to the animal as opposed to the environment.

Through a human interface, the controller is programmed with certain information about the animal, that information being either a general profile (e.g., condition) or animal specific information such as age and weight. The information becomes part of the analysis parameters that define or modify the evaluation criteria and/or the calculations used to make a condition evaluation. When the result of the evaluation exceeds a threshold value or otherwise meets specified criteria, a condition warning or alert is generated. The results obtained using the same physiological and environmental conditions will often vary based upon the animal profile information.

A condition indicator includes a human interface that is responsive to the controller and produces a response indicative of the condition of the animal. The response can be generated locally at the unit worn by the animal or the unit can be equipped with a transmitter to provide a remote condition notification.

### BRIEF DESCRIPTION OF THE SEVERAL VIEWS OF THE DRAWINGS

The above-mentioned features of the invention will become more clearly understood from the following detailed description of the invention read together with the drawings in which:

FIG. 1 is a representation of an animal carrying an animal condition monitor according to one embodiment of the present invention;

FIG. 2 is schematic representation of one embodiment of a circuit implementing the animal condition monitor;

FIG. 3 is a block diagram of one embodiment of the animal condition monitor;

FIG. 4 is a flow chart of one embodiment of the method applied by one embodiment of the animal condition monitor;

FIG. 5 is an exemplary portion of a look-up table determining the result for a specific set of conditions using the young and old animal profile;

FIG. 6 is an exemplary portion of a look-up table determining the result for a specific set of conditions using the house pet profile;

FIG. 7 is an exemplary portion of a look-up table determining the result for a specific set of conditions using a sporting dog profile; and

FIG. 8 is a block diagram of an alternate embodiment of the animal condition monitor having a remote notification unit.

### DETAILED DESCRIPTION OF THE INVENTION

One embodiment of an animal condition monitor is shown and described at 100 in the figures. By way of example, the disclosed animal condition monitor is an animal heat exhaustion monitor 100. The animal heat exhaustion monitor 100 measures diagnostic parameters from the animal and the environment and determines whether the parameters indicate that the animal is at risk of heat exhaustion. When a heat exhaustion risk is indicated, the animal heat exhaustion monitor 100 provides notification of the risk.

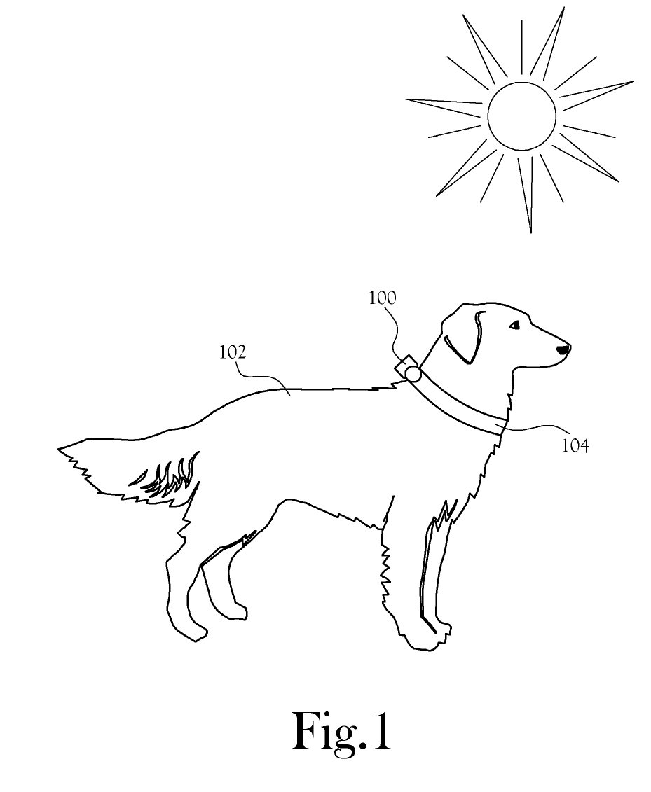

FIG. 1 depicts an animal 102, specifically a dog, wearing the animal heat exhaustion monitor 100 of the present invention. In the illustrated embodiment, the animal heat exhaustion monitor 100 is carried by a collar 104 worn by the animal 102. Those skilled in the art will recognize that carriers other than collars can be used without departing from the scope and spirit of the present invention. Further, although illustrated as being carried proximate to the spinal cord, the animal heat exhaustion monitor can be placed in other positions without departing from the scope and spirit of the present invention.

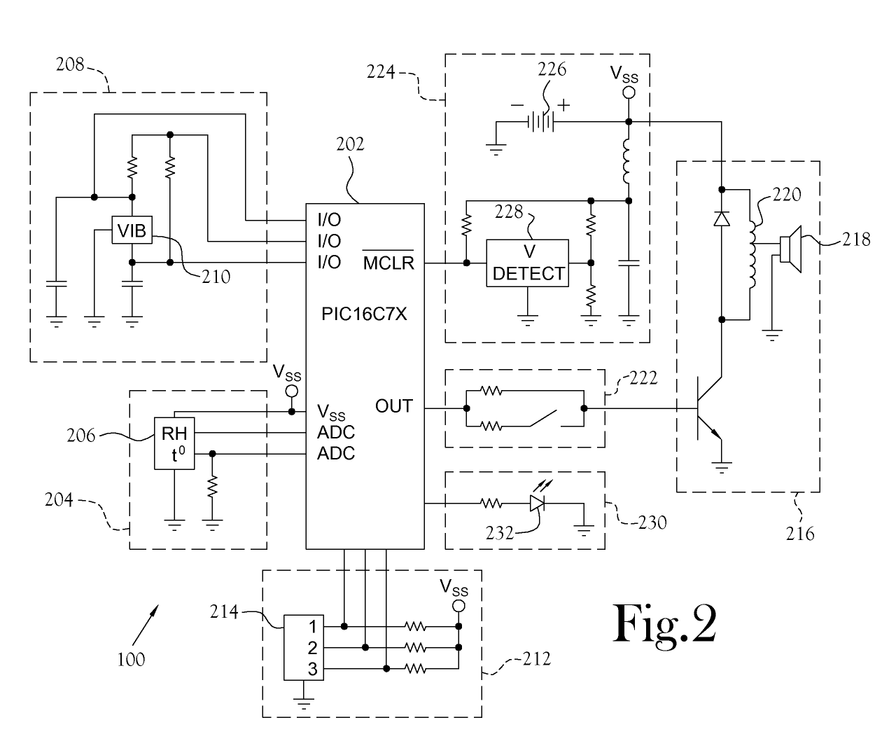

FIG. 2 is a schematic of one embodiment of an animal heat exhaustion monitor 100 according to the present invention. Central to the animal heat exhaustion monitor 100 is a controller 202. The controller 202 receives inputs from sensors that provide information about the environmental conditions, the activity level of the animal 102, and the age and physical condition/characteristics of the animal 102. Using the inputs, the controller 202 evaluates the risk of heat exhaustion and produces an output that indicates the relative risk of heat exhaustion. One suitable implementation of the controller 202 uses a microcontroller from the PIC16C7X series manufactured by Microchip Technology, Inc., as illustrated in FIG. 2.

The animal heat exhaustion monitor 100 includes an environmental condition sensor 204 in communication with the microcontroller 202. The environmental condition sensor 204 is capable of measuring at least the ambient temperature. In the illustrated embodiment, the environmental condition sensor 204 includes a relative humidity and temperature sensor 206 such as the HS-2000V manufactured by Precon. The relative humidity and temperature sensor 206 produces a ratiometric first voltage output corresponding to the percent relative humidity detected and a ratiometric second voltage output corresponding to the measured temperature. Conveniently, the microcontroller 202 selected for this embodiment includes four analog-to-digital converter (ADC) channels. The analog voltages produced by the relative humidity and temperature sensor 206 are converted to digital values for use by the microcontroller 202. However, one skilled in the art will appreciate alternate mechanisms for interfacing the environmental condition sensor 204 with the controller 202.

Both the temperature sensor and the relative humidity sensor produce an output voltage that corresponds to a temperature and a relative humidity, respectively. One skilled in the art will appreciate the various methods for producing a meaningful comparison of the temperature and humidity outputs to reference values. Where the analog voltages are digitized, as described above, the digital values are easily scaled to actual temperature and humidity values. These scaled values are then compared to specific temperature and humidity thresholds. Alternatively, the temperature and humidity thresholds are scaled to match the output voltages.

The animal heat exhaustion monitor 100 also includes an activity sensor 208 in communication with the microcontroller 202. In one embodiment, the activity sensor 208 includes a vibration sensor 210. One suitable vibration sensor 210 is a ball tilt sensor. Other suitable vibration sensors include accelerometers and piezoelectric sensors. The vibration sensor 210 produces an output when the animal heat exhaustion monitor 100 is moved. In one embodiment, the signal produced by the vibration sensor 210 is supplied to the microcontroller 202 and the microcontroller counts the length of time that the vibration sensor 210 is active. The microcontroller 202 of the present embodiment includes a timer, which allows the timing to be measured internally. Alternatively, an external timer may be used. In another embodiment, the microcontroller 202 determines the level of activity by measuring the rate of vibration detected by the vibration sensor 210.

A profile selector 212 in communication with the microcontroller 202 provides an interface that allows the user to select a profile for the animal 102. The selected profile determines the values at which the microcontroller 202 produces alerts. In the illustrated embodiment, a switch 214, such as a binary-coded decimal switch, allows selection of one of multiple profiles, which allow selection of a profile corresponding to various factors such as the age, health, and conditioning of the animal which are represented by profiles such an athletic/sporting animal, a young or elderly animal, and an indoor animal. In other embodiments, the profile information is less general and contains information specific to the animal 102, such as weight, age, percentage time indoors, and conditioning. In another embodiment, the profile selector 212 is a data input keypad allowing direct entry of threshold information.

A heat exhaustion indicator 216 responsive to the microcontroller 202 produces an indication to a human that the animal is at risk of heat exhaustion or of heat stroke. In the illustrated embodiment, the heat exhaustion indicator 216 includes a speaker 218 connected to a transformer 220 for producing an audible alert. The audible alert varies with the severity of the risk. A volume selector 222 allows adjustment of the volume level of the audible alert. In the illustrated embodiment, the volume selector 222 provides two volume settings.

The power supply 224 of the animal heat exhaustion monitor 100 includes a portable power source 226, such as a battery, to produce the supply voltage Vss. A voltage detector 228 is used to implement a brown-out eliminator circuit that handles under voltage situations and prevents damage to the components of the animal heat exhaustion monitor 100. In the event that the voltage of the battery 226 drops below a selected level that is sufficient to reliably drive the circuitry of the animal heat exhaustion monitor 100, a device status indicator 230 provides an indication of the low battery condition. In the illustrated embodiment, the device status indicator 230 includes a lamp 232, such as a light emitting diode, to provide a visual indication of the low battery condition.

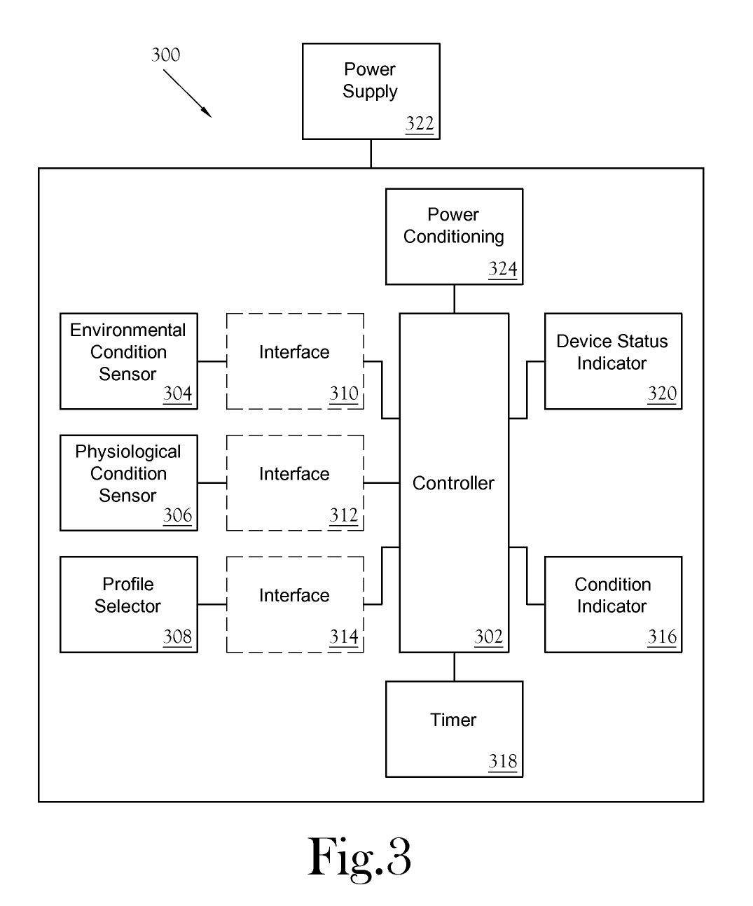

While one specific embodiment has been shown, other embodiments and variations are contemplated by the present inventor. FIG. 3 is a block diagram of a general embodiment of an animal condition monitor 300. The controller 302 provides the processing and control to evaluate the input conditions and generate an indication in the event that the risk to the animal of a condition such as heat exhaustion exceeds a selected level. In addition to embodiment described above that uses a microcontroller, other controller implementations are available for producing an output based upon a set of inputs.

One input to the controller 302 is generated from an environmental condition sensor 304. The previously discussed embodiment utilizes both humidity sensing and temperature sensing. In another embodiment, only temperature is monitored using a device such as the KTY81-2 series silicon temperature sensor from Philips Electronics N.V. Other environmental sensors are available to measure various parameters. The type and number of environmental condition sensors used is limited only by the design considerations for the animal condition monitor 300 which include the size, weight, and cost of the unit, the relevance of environmental condition to monitored condition (e.g., heat exhaustion), and the complexity of the calculations or software.

Another input to the controller 302 comes from one of the available physiological condition sensors, such as the activity sensor 306 previously detailed. As shown above, a vibration sensor is a suitable activity sensor 306 for indicating when the animal 102 is active. In one embodiment, the output of the activity sensor 306 has two states with a first state indicating activity and a second state indicating no activity. In order to establish a stable output, a sample-and-hold circuit is used and the length of time that the output indicates activity is measured. In another embodiment, the activity sensor 306 produces an output signal containing frequency information. The frequency information is indicative of the intensity of the activity undertaken by the animal. For example, walking produces a relatively low-frequency vibration while running and jumping produces a vibration at a higher frequency. Considering both the duration and the intensity of the activity through an appropriate analysis offers the opportunity to evaluate the risk of heat exhaustion with greater certainty.

In other embodiments, other physiological condition sensors measure physical statistics of the animal. Various embodiments employ heart rate sensors, respiration sensors monitoring the respiration rate or the respiration output of the animal, and surface or basal/core body temperature monitors. It will be appreciated by those skilled in the art that either the surface body temperature or the core body temperature can be estimated using the other temperature to within a reasonable degree of certainty based on known relationships between basal and surface body temperatures. As should be appreciated from the description of the present invention, in the context of the present invention, the term physiological condition sensor refers to sensors that obtain information related to the animal as opposed to the environment. Thus, although not a measurement of a physiologic process in the animal's body (e.g., heart rate, blood pressure, etc.), activity level, movement time, speed, distance, and other events or variables associated with the animal are deemed to be physiological conditions.

The manual input to the controller 302 is provided by a user through the profile selector 308. As previously mentioned, the profile selector 308 sets the profile used in the analysis. Each profile is associated with set of analysis parameters. In one embodiment, the analysis parameters are factors that modify the calculations and the results are compared against a fixed scale. When the result of the calculations exceeds a selected value in the fixed scale, a heat exhaustion warning is generated. For the same activity level and environmental conditions, selecting an athletic animal profile would result in a lower score than if an elderly animal profile is used. In another embodiment, the calculations are fixed but the scale is adjusted. The score based upon identical activity level and environmental conditions would be the same for all animals, but selecting an athletic animal profile would require a higher score to be calculated, when compared to an elderly animal profile, in order to generate a heat exhaustion alert.

Depending upon the components used for the controller 302, the environmental condition sensor 304, the activity sensor 306, and the profile selector 308 optional interfaces 310, 312, 314 are employed. For example, when digital processing of the outputs from analog sensors and selectors is required, analog-to-digital converters are required. Such interface may be integrated in the sensor, the selector, or the controller, or require a non-integrated solution, such as a separate analog-to-digital converter. It is deemed to be within the purview of one skilled in the art to properly interface the inputs to the controller 302.

A condition indicator 316 is a human interface that is responsive to the controller 302 and produces a response indicative of the condition of the animal. In the embodiment of FIG. 2, the heat exhaustion indicator produces an audible alarm. In another embodiment, the heat exhaustion indicator produces a visual alert using one or more lamps or other visual indicators. In another embodiment, additional responses are generated by providing warnings and alerts that have an intensity that corresponds to the severity of the alarm. In general, variations in the volume, tone, beat, frequency, rate, color, pattern, or other characteristics are used to indicate the relative risk level. For example, the color and/or number of lamps vary with the severity of the heat exhaustion risk or the number of beeps in sound burst varies to indicate the risk.

The general embodiment of the animal condition monitor 300 includes a timer 318 that allows temporal relationships to be determined. Often, it is useful to determine the time between occurrence of events/conditions measured by any of the associated environmental condition sensors 304 or the physiological condition sensors 306.

The general embodiment of the animal condition monitor 300 further includes a device status indicator 320. In many embodiments, the device status indicator 320 is a simple battery level/low battery indicator. In other embodiments, advanced status information, such as the malfunction of a sensor, is implemented based upon the response of the sensor received by the controller. Alternatively, certain sensors are capable of generating specific error signals that are read and interpreted by the controller. Alternatively, one embodiment of the animal condition monitor 300 evaluates the sensor output for anomalies and generates fault codes to indicate that one or more sensors appear to be malfunctioning. For example, if consecutive temperature readings show a large and generally unstable variance, the controller 302 indicates that the temperature sensor 306 is not operating properly. Depending upon the design considerations, the device status indicator 320 is implemented using audible or visual indicators.

For completeness, the animal condition monitor 300 of FIG. 3 includes components that are either common to or necessary to electronic devices. For example, electronic devices necessarily include a power supply 322 which commonly is interfaced to the remainder of the circuitry through a power conditioning circuit 324. The power supply 322 typically will be in the form of a battery for portability. However, one skilled in the art will recognize that any suitable power supply may be used without departing from the scope and spirit of the present invention. The block diagram of FIG. 3 generally illustrates the components making up the animal condition monitor 300. It is not intended to show all interconnections. For example the power supply 322 is shown generally connected to the animal condition monitor. One skilled in the art would appreciate that the power supply would be connected to each of the various components, either directly or indirectly.

FIG. 4 is a flow chart of one embodiment of the method 400 associated with the specific embodiment animal heat exhaustion monitor 100. In the illustrated embodiment, the method 400 begins with the step of initialization 402 of the animal heat exhaustion monitor 100. Initialization places the animal heat exhaustion monitor 100 in a known state. Typical processes for initialization 402 include setting or clearing variables, diagnostics such as a power on self test routine. Such procedures are implementation specific and will be appreciated by those skilled in the art.

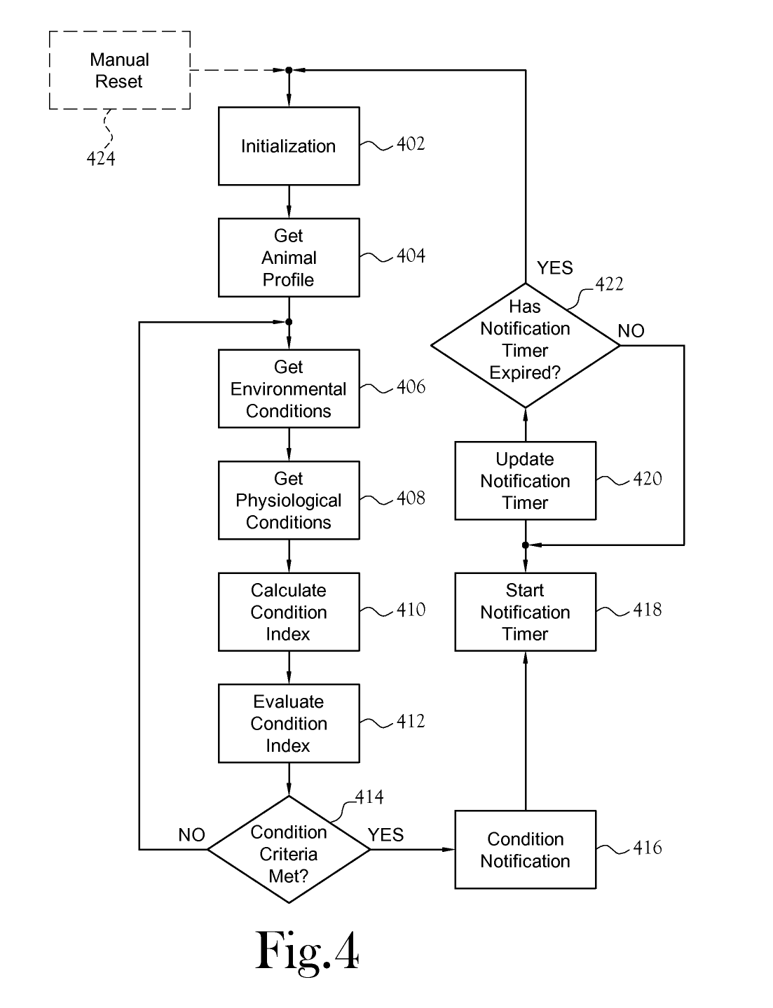

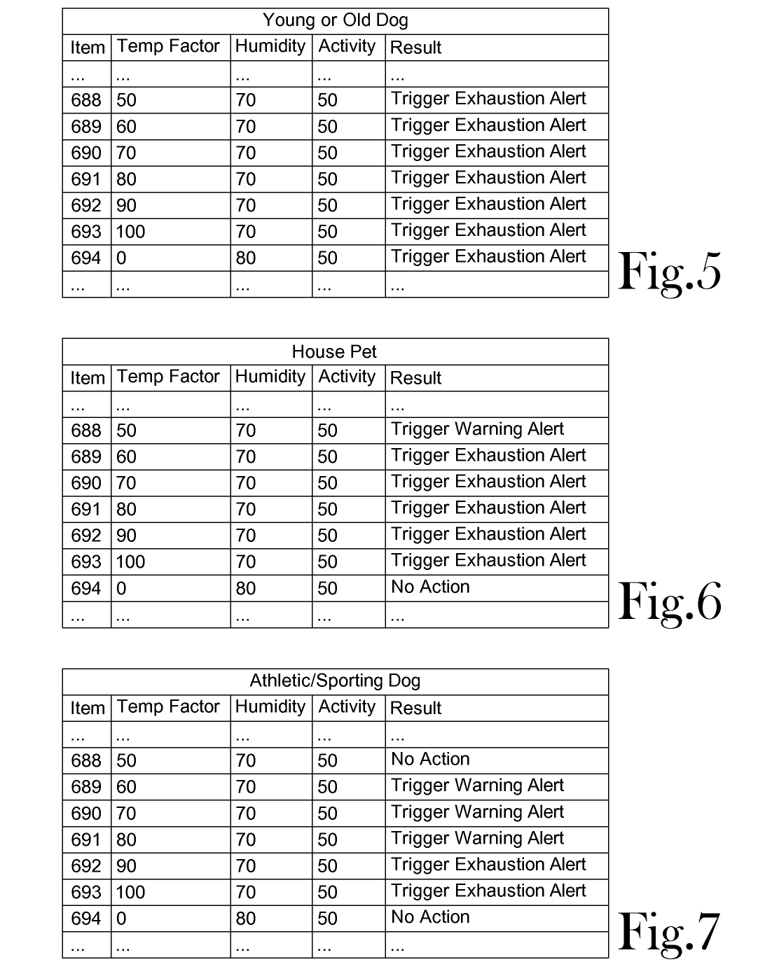

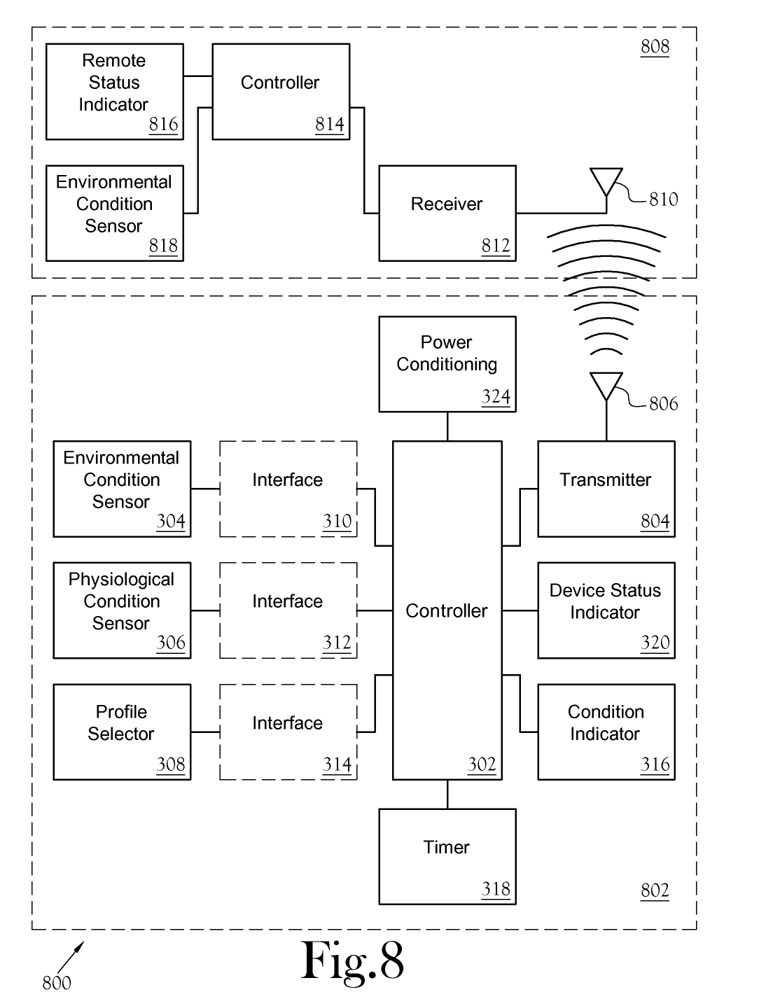

Next, the animal heat exhaustion monitor 100 reads and selects an animal profile 404 from the profile selector 308. Providing differing animal profiles allows the alerts conditions to be varied to account for the factors including the age, training, and lifestyle of the animal. In one embodiment, one of a number of preset look-up tables is used based upon the selected animal profile. An exemplary portion of the look-up tables for puppies and elderly dogs, for house pets, and for sporting dogs are illustrated in FIGS. 5, 6, and 7, respectively.

Once the animal heat exhaustion sensor has been configured, the main portion of the method begins with measuring the ambient environmental conditions 406. As previously described, one embodiment of the animal heat exhaustion monitor 100 measures the temperature and the humidity. In addition to the environmental conditions, the animal heat exhaustion monitor 100 monitors the activity level (physiological condition) of the animal 408.

Using the environmental conditions and the activity level of the dog, an exhaustion index is derived 410. The environment conditions, the activity level of the dog, and the alert conditions must be correlated to have meaning. Again, the available correlation procedures are implementation specific and will be appreciated by those skilled in the art. Generally, calculation of the exhaustion (condition) index refers to calculating, correlation, cross-referencing, indexing, scaling, grouping, or looking-up a value or values that can be used to evaluate the condition. Next the exhaustion index is evaluated 412 against a threshold valve or a reference. If the necessary exhaustion conditions are met 414, an exhaustion notification is generated 416 and a notification timer is activated 418. The notification timer is updated 420 until the notification timer expires 422. After the expiration of the notification timer, the main portion of the method continues again starting with initialization 402, thereby resetting the condition notification. If the exhaustion conditions are not met, the main portion of the method continues again starting with obtaining the environmental conditions 406. In one embodiment, an opportunity to manually reset the animal condition monitor to the initialization point is provided 424. One skilled in the art will appreciate that the initialization step need not be repeated in all embodiments.

For the embodiment utilizing a look-up table associated with the selected animal profile, the general method described above operates as follows. The temperature index is compared to the temperature values in the look-up table. The rows of the look-up table where the temperature value equals the temperature index are selected as a first subset of the of the look-up table. Next, the relative humidity index is compared to the relative humidity values in the first subset of the look-up table. The rows of the first subset of the look-up table where the relative humidity value equals the relative humidity index are selected as a second subset of the look-up table. Finally, the activity index is compared to the activity values in the second subset of the look-up table. The row of the second subset of the look-up table where the activity value equals the activity index is selected as the result of the exhaustion index comparison. This is the row that matches all three index values and provides a corresponding response.

In one embodiment, the available responses include no response, an exhaustion warning, and an exhaustion alert. It will be appreciated by one skilled in the art that any specific implementation can employ other responses and/or responses with increased precision.

In another embodiment, the repeating portion of the method updates the animal profile 404 allowing variations in the animal profile to be made without reinitializing the animal heat exhaustion monitor 100. The first embodiment places emphasis on profile security by, in effect, ignoring subsequent changes to the profile either accidentally or on purpose. The alternate embodiment emphasizes flexibility by allowing on-the-fly changes to the animal profile.

In an alternate embodiment, calculation of the exhaustion (condition) index involves converting either the threshold values or the measured condition values to allow for meaningful comparison. In the context of converting threshold values, the set points for exhaustion warning conditions might be input in human terms, i.e., 90 degree Fahrenheit, but the value would be implemented as a voltage selected to reflect the output voltage of the sensor corresponding to that temperature. Conversely, if the comparison is performed in terms of temperature, the output of the sensor is converted into the temperature corresponding to the output voltage and compared against the temperature number. Similarly, scaling is an alternate embodiment where the results are normalized to a range of values, for example, between 0 and 100. Thus, where the output voltage of a temperature sensor represents a range of 130 degrees Fahrenheit (−20 to 110 degrees), the values can be scaled, either linearly or nonlinearly, into a hundred point scale. These types of conversions/scaling are well understood by those skilled in the art.

In a still further embodiment, the various measured conditions (environmental and physiological) values are evaluated in a formula that allows the various condition values to be weighted, if desired, to create an index that can be compared to a discrete threshold value. For example, the temperature value, the humidity value, and the activity value are variables in an equation that produces an index with a value between 0 and 100. The comparison thresholds in this example are that any score above 60 produces a warning and any score above 70 produces an alert and the temperature accounts for approximately 33% of the index, the humidity accounts for approximately 50% of the index, and the activity accounts for the remaining percentage, or approximately 17%. Further, the cadence measured using the ball tilt sensor is scaled by a selected factor to be commensurate with the other values being combined. However, the present invention contemplates a platform for condition monitoring and not a specific test for any particular condition. Obviously, the relative weights attributed to the variables affect the specified result. What conditions warrant warnings of a potentially dangerous condition are subjective and, therefore, the particular set of conditions triggering a warning are implementation specific. One embodiment might be very cautious in providing a warning while another might be very aggressive.

FIG. 8 is a block diagram of an alternate embodiment of the animal condition monitor 800 including a remote notification unit 808. In most respects, the collar mounted unit 802 worn by the animal is similar to the embodiment described with respect to FIG. 3. Notable exceptions include the addition of a transmitter 804 and a corresponding antenna 806. It should be recognized that the power supply 322 has been omitted from the illustration but remains a necessary part of electronic equipment. The remote notification unit generally includes an antenna 810 and the corresponding receiver 812, a controller 814, a status indicator 816, and, optionally, an environmental condition sensor 818.

The principle of operation remains the generally the same, i.e., conditions are monitored and evaluated and notification of the condition is provided when necessary but the condition notification occurs on a remote device, in place of or in addition to the local communication notification. In the embodiment of FIG. 3, all monitoring, evaluation, and notification occurs in the unit worn by the animal. In one version of the embodiment of FIG. 8, monitoring, evaluation, and notification occur in the unit worn by the animal with remote notification also occurring in the remote unit. In another version, monitoring and evaluation occur in the unit worn by the animal with remote notification occurring only in the remote unit. In still another version, monitoring occurs in the unit worn by the animal but the measured conditions are transmitted to the remote unit where evaluation and notification occurs. Finally, using the optional environmental condition sensor in the remote notification unit, monitoring is split between the remote notification unit and the unit worn by the animal with the ambient/environmental conditions being measured by the remote notification unit and the physiological conditions being measured by the unit worn by the animal and transmitted to the remote notification unit for evaluation and notification.

In one version of the embodiment of FIG. 8, the remote notification unit is a portable unit that can be carried or worn by a person desiring to receive notification. In an alternate version, the remote notification unit is a base station generally designed to be located or installed in a stationary location. Certainly, the stationary remote notification unit is transportable as needed.

The transmitter and receiver shown in FIG. 8 are intended to be representative of general communication devices and are intended to encompass components such as encoders/decoders, modulators/demodulators, amplifiers, filters, etc., that are useful or necessary for the implementation of communcation. In one embodiment, communication is through radio frequency communications. In another embodiment, communication is accomplished using cellular communication technology. In a still further embodiment, magnetic or electromagnetic fields are used for communication. The choice of communication technologies is largely dependent on range and power requirements/limitations. In the various embodiments, the communication between the transmitter and the remote device is implemented using radio frequency, infrared, ultrasonic, magnetic fields, Bluetooth®, cellular, or similar types of communication technologies and is modulated or coded as necessary to convey the condition information.

The foregoing description discloses, in one embodiment, an animal condition monitor designed specifically for monitoring the condition of heat exhaustion. In a broader embodiment, the animal condition monitor measures other characteristics of an animal and/or the environment to monitor another specified condition. The block diagram of FIG. 3 illustrates one suitable structure of a general platform for monitoring the condition of an animal based upon the implemented sensors. Although described in terms of monitoring for the condition of heat exhaustion, the method of FIG. 4 is easily extrapolated or generalized to monitor other similar conditions (e.g., hypothermia, hyperthermia, dehydration, etc).

The animal condition monitor of the present invention has been disclosed in the description and figures. The animal condition monitor utilizes one or more sensors that measure conditions of the animal (i.e., physiological conditions) and the environment ambient to the animal (i.e., environmental conditions). A profile of the animal provides information relating to the age and/or condition of the animal. A control circuit reads the profile information and the information from the sensors and provides notification when the information from the sensors and the profile indicates that the animal is at risk. An alert is generated locally, remotely, or in both locations.

While the present invention has been illustrated by description of several embodiments and while the illustrative embodiments have been described in detail, it is not the intention of the applicant to restrict or in any way limit the scope of the appended claims to such detail. Additional modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicants general inventive concept.

Having thus described the aforementioned invention, what is claimed is:

1. An animal condition monitor for use with an animal, said animal condition monitor comprising:
an environmental condition sensor measuring an environmental condition ambient to said environmental condition sensor and producing a first output corresponding to said environment condition;
a physiological condition sensor carried by the animal, said physiological condition sensor measuring a physiological condition of the animal and producing a second output corresponding to said physiological condition;
a controller in communication with said environmental condition sensor and said physiological condition sensor, said controller evaluating said first output and said second output against a set of criteria indicative of a selected condition to produce a result output; and
an output device in communication with said controller providing an indication of said result output.

2. The animal condition monitor of claim 1 further comprising a profile selector in communication with said controller, said profile selector allowing an operator to input profile information about the animal.

3. The animal condition monitor of claim 2 wherein said profile information is selected from the group of consisting of age of the animal, conditioning of the animal, and health of the animal.

4. The animal condition monitor of claim 1 wherein said output device further comprises a visual indicator in communication with said controller.

5. The animal condition monitor of claim 1 wherein said output device further comprises an audible indicator in communication with said controller.

6. The animal condition monitor of claim 1 further comprising a remote indicator comprising a receiver and an indicator, wherein said output device comprises a transmitter in communication with said controller, said transmitter producing a communication signal receivable by said receiver, said communication signal communicating said result output to said remote indicator.

7. The animal condition monitor of claim 6 wherein said remote indictor further comprises a visual indicator in communication with said receiver.

8. The animal condition monitor of claim 6 wherein said remote indicator further comprises an audible indicator in communication with said receiver.

9. The animal condition monitor of claim 1 wherein said environmental condition sensor is selected from the group consisting of a temperature sensor and a humidity sensor.

10. The animal condition monitor of claim 1 wherein said physiological condition sensor is selected from the group consisting of an activity level sensor, a pedometer, a thermometer, and a heart rate monitor.

11. The animal condition monitor of claim 1 wherein said set of criteria comprises a look-up table containing results for combinations of possible values of at least said first output and said second output, one of said results corresponding to said first output and said second output being selected as said result output.

12. The animal condition monitor of claim 1 wherein said set of criteria comprises at least one threshold value.

13. The animal condition monitor of claim 1 wherein said environmental condition sensor is a temperature sensor, said first output corresponds to a temperature ambient to said animal condition monitor, said physiological condition sensor is an activity level sensor, and said second output corresponds to movements of the animal, said animal condition monitor further comprising a humidity sensor in communication with said controller, said humidity sensor producing a third output corresponding to a humidity ambient to said animal condition monitor, said controller evaluating said first output, said second output, and said third output against said set of criteria.

14. An animal condition monitor for use with an animal, said animal condition monitor comprising:
an environmental condition sensor carried by the animal, said environmental condition sensor measuring an environmental condition ambient to said environmental condition sensor and producing a first output corresponding to said environment condition;
a physiological condition sensor carried by the animal, said physiological condition sensor measuring a physiological condition of the animal and producing a second output corresponding to said physiological condition;
a profile selector allowing an operator to input profile information about the animal;
a controller in communication with said environmental condition sensor, said physiological condition sensor, and said profile selector, said controller evaluating said first output and said second output against a set of criteria corresponding to said profile information, said set of criteria indicative of a selected condition to produce a result output; and
an output device in communication with said controller providing an indication of said result output.

15. The animal condition monitor of claim 14 wherein said environmental condition sensor is selected from the group consisting of a temperature sensor and a humidity sensor.

16. The animal condition monitor of claim 14 wherein said physiological condition sensor is selected from the group consisting of an activity level sensor, a pedometer, a thermometer, and a heart rate monitor.

17. The animal condition monitor of claim 14 wherein said set of criteria comprises a look-up table containing results for combinations of possible values of at least said first output and said second output, one of said results corresponding to said first output and said second output being selected as said result output.

18. The animal condition monitor of claim 14 wherein said set of criteria comprises at least one threshold value.

19. The animal condition monitor of claim 14 wherein said output device is in remote communication with said controller.

20. The animal condition monitor of claim 14 further comprising:
a transmitter in communication with said controller, said transmitter being carried by the animal, said transmitter sending an alert signal in response to said result output;
a remote notification unit housing;
wherein said output device is carried by said remote notification unit housing; and
a receiver in communication with said transmitter and said output device, said receiver being carried by said remote notification unit housing, said receiver receiving said alert signal and activating said output device.

21. The animal condition monitor of claim 14 wherein said controller is in remote communication with said environmental condition sensor and said physiological condition sensor.

22. The animal condition monitor of claim 14 further comprising:
a transmitter in communication with said environmental condition sensor and said physiological condition sensor, said transmitter being carried by the animal, said transmitter sending said first output and said second output;
a remote notification unit housing;
wherein said controller is carried by said remote notification unit housing;
wherein said output device is carried by said remote notification unit housing; and
a receiver in communication with said transmitter and said controller, said receiver being carried by said remote notification unit housing, said receiver receiving said first output and said second output and passing said first output and said second output onto said controller.

23. The animal condition monitor of claim 14 wherein said controller is in remote communication with said physiological condition sensor.

24. The animal condition monitor of claim 14 further comprising:
a transmitter in communication with said physiological condition sensor, said transmitter being carried by the animal, said transmitter sending said second output;
a remote notification unit housing;
wherein said environmental condition sensor is carried by said remote notification housing;
wherein said controller is carried by said remote notification unit housing;
wherein said output device is carried by said remote notification unit housing; and
a receiver in communication with said transmitter and said controller, said receiver being carried by said remote notification unit housing, said receiver receiving said second output and passing said second output to said controller.

25. A method for providing a notification of a condition of an animal using an animal condition monitor including an environmental condition sensor, a physiological condition sensor, a controller in communication with the physiological condition sensor and the environmental condition sensor, and a condition indicator in communication with said controller, said method comprising the steps of:
(a) measuring at least one environmental condition proximate to an animal using said environmental condition sensor;
(b) measuring at least one physiological condition of the animal using said physiological condition sensor;
(c) providing at least one set of criteria associated with a condition of the animal;
(d) evaluating said at least one environmental condition and said at least one physiological condition against said at least one set of criteria; and
(e) providing a notification when said step of evaluating indicates a condition of the animal.

26. A method for providing a notification of a condition of an animal using an animal condition monitor including an environmental condition sensor, a physiological condition sensor, a profile selector, a controller in communication with the physiological condition sensor, the environmental condition sensor, and the profile selector, and a condition indicator in communication with said controller, said method comprising the steps of:
(a) entering profile information about the animal using the profile selector;
(b) measuring at least one environmental condition proximate to an animal using the environmental condition sensor;
(c) measuring at least one physiological condition of the animal using the physiological condition sensor;
(d) providing at least one set of criteria associated with a condition of the animal;
(e) selecting evaluation criteria from said at least one set of criteria based upon said profile information;
(f) evaluating said at least one environmental condition and said at least one physiological condition against said evaluation criteria; and
(g) providing a notification when said step of evaluating indicates a condition of the animal.

27. A method for providing a notification of a condition of an animal using an animal condition monitor including an environmental condition sensor, a physiological condition sensor, a profile selector, a controller in communication with the physiological condition sensor, the environmental condition sensor, and a threshold selector, and a condition indicator in communication with said controller, said method comprising the steps of:
(a) entering threshold information about the animal using the threshold selector;
(b) measuring at least one environmental condition proximate to an animal using the environmental condition sensor;
(c) measuring at least one physiological condition of the animal using the physiological condition sensor;
(d) providing at least one set of criteria associated with a condition of the animal;
(e) selecting evaluation criteria from said at least one set of criteria based upon said threshold information;
(f) evaluating said at least one environmental condition and said at least one physiological condition against said evaluation criteria; and
(g) providing a notification when said step of evaluating indicates a condition of the animal.
